(19) United States
(12) Patent Application Publication
(10) Pub. No.: US 2011/0251584 A1
(43) Pub. Date: Oct. 13, 2011

(54) METHOD AND APPARATUS FOR DELIVERING CONTINUOUS INTRAVENOUS INFUSION OF METOPROLOL

(76) Inventor: Umesh Khot, Indianapolis, IN (US)

(21) Appl. No.: 12/756,360

(22) Filed: Apr. 8, 2010

(51) Int. Cl. A61M 5/168

(52) U.S. Cl. 604/504; 604/66; 604/503

(57) ABSTRACT

A system for automatically providing a continuous flow of metoprolol intravenously to a patient, including a supply of metoprolol solution, a pump connected in fluidic communication with the supply of metoprolol solution, an electronic controller operationally connected to the pump, a vascular access device connected in liquid communication to the pump, and at least one sensor operationally connected to the pump. The pump provides a continuous flow of metoprolol through the vascular access device and the at least one sensor provides data regarding a patient's physical condition to the electronic controller. The electronic controller may control the pump to vary the flow rate of metoprolol in response to data from the at least one sensor and may generate an alarm signal in response to data from the sensor falling outside of predetermined parameters.

### TECHNICAL FIELD

The invention relates generally to the field of medicine and, specifically, to a method and apparatus for providing the continuous infusion of metoprolol to a patient.

### BACKGROUND

Metoprolol is a one of the most commonly prescribed member of a class of drugs called beta-blockers (also known as beta-adrenergic blocking agents or beta-receptor antagonists). Beta-blockers work by blocking the effects of catecholamines such as epinephrine on the beta-receptors located within the heart, kidney, and other organ systems. Beta-blockers also reduce sympathetic nervous system activity by decreasing the release of the hormone renin from the kidney and by reducing the amount of sympathetic nervous system activity originating in the brain. The resulting effect on the heart is a decrease in heart rate (negative chronotropic effect) and a decrease in the strength of contraction of the heart (negative inotropic effect). The drugs have been shown to help decrease blood pressure, stabilize heart rhythm, decrease the frequency of heart-related chest pains (i.e. angina), decrease the risk of heart attack, and decrease the risk of sudden cardiac death. Clinically, beta-blockers such as metoprolol are currently highly recommended and widely used for the treatment of high blood pressure, stable/unstable angina (heart-related chest pains), acute myocardial infarction (heart attack), coronary artery disease, congestive heart failure, and atrial fibrillation (an irregular rhythm of the heart).

Given its widespread utilization, many patients are taking oral metoprolol prior to being hospitalized. Current medical practice recommends that patients be continued on their metoprolol during their hospital stay as discontinuing the drug can lead to life-threatening complications including severe hypertension, heart rhythm problems, myocardial infarction, and death. In addition, during hospitalization, many patients develop one of the above medical conditions that would benefit from metoprolol therapy. Thus, there is a need within two large populations of patients to continue or start taking metoprolol therapy during their hospitalization.

Metoprolol is typically administered orally. Unfortunately, despite a documented clinical need for the drug, many patients cannot take oral metoprolol during hospitalization. These include the following groups of patients:
1. Patients who are NPO (nil per os-no oral intake) for any reason.
2. Patients who are unable to take oral medications due to dysphagia, swallowing difficulties, mental status changes, or stroke.
3. Patients who are critically ill (i.e. on a ventilator) whose condition make it not possible or not safe to administer oral medications.
4. Patients who are unable to take oral medications due to abnormalities of their gastrointestinal tract (i.e. ileus, bowel obstruction, recent abdominal surgery, pancreatitis).
5. Patients who recently underwent surgery and are unable to take oral medications during the early-postoperative time period.

Thus, there is a need for an alternative to oral metoprolol for these patients. The present invention addresses this need.

### SUMMARY

The present invention relates to a system and method for providing metoprolol to patients. One object of the present invention is to provide an improved means for administering metoprolol to patients. Related objects and advantages of the present invention will be apparent from the following description.

### BRIEF DESCRIPTION OF THE DRAWINGS

FIG. 1 is a perspective view of a metoprolol delivery system according to a first embodiment of the present novel technology.

FIG. 2 is a perspective view of a metoprolol delivery system according to a second embodiment of the present novel technology.

FIG. 3 is a perspective view of a metoprolol delivery system according to a third embodiment of the present novel technology.

### DESCRIPTION OF THE PREFERRED EMBODIMENTS

For the purposes of promoting an understanding of the principles of the invention and presenting its currently understood best mode of operation, reference will now be made to the embodiments illustrated in the drawings and specific language will be used to describe the same. It will nevertheless be understood that no limitation of the scope of the invention is thereby intended, with such alterations and further modifications in the illustrated device and such further applications of the principles of the invention as illustrated therein being contemplated as would normally occur to one skilled in the art to which the invention relates.

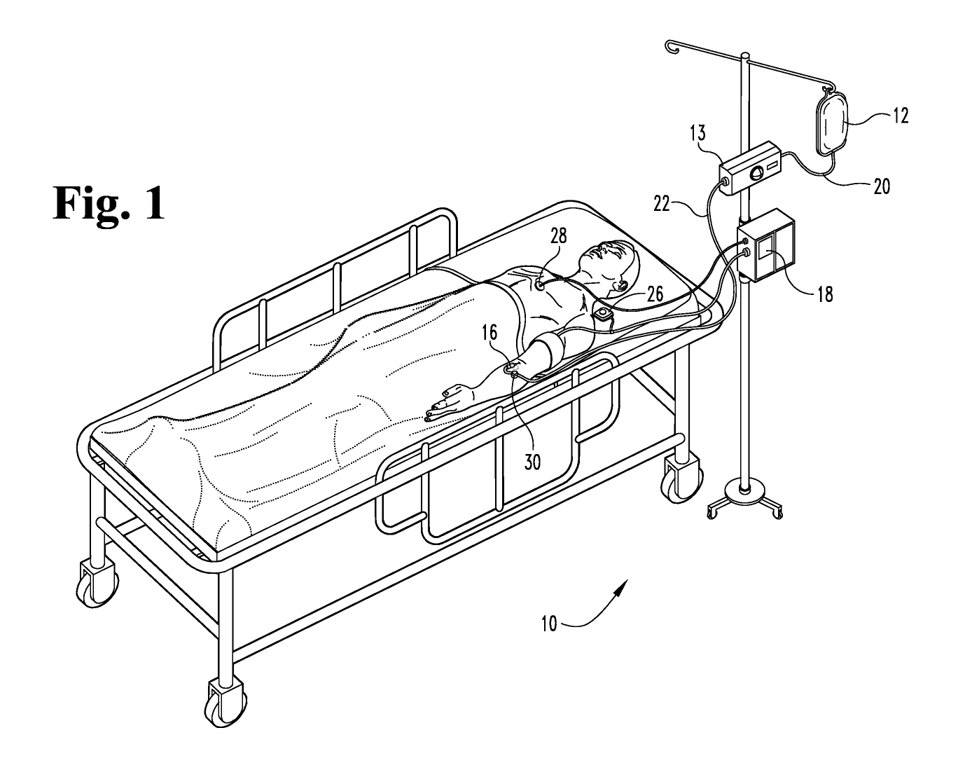

As illustrated in FIG. 1, a first embodiment of the present novel technology relates to a system 10 for the continuous infusion of metoprolol to a patient. The system 10 includes a metoprolol supply 12. Metoprolol source 12 is typically a 50 or 100 mg bag, although bags or containers of other sizes may be selected as desired. The metoprolol source 12 may contain a standard concentration of metoprolol, akin to that found in 5 mg single-dosage vials (typically 1 mg/ml), or, alternately, may contain a metoprolol solution having a lower (or greater) metoprolol concentration. The metoprolol source is in liquid communication with an infusion device 13. The infusion device 13 is typically a pump, but may be a stopcock, electronically controlled valve, or the like. The infusion device 13 is connected in liquid communication with catheter 16 or like device for insertion into communication with a patient's circulatory system. A heart rate sensor 26 is connected in electric communication to a telemetry system 18, typically either via direct wire connection or wirelessly to a remotely located telemetry system 18. The patient's blood pressure is periodically measured, either via manual or automatic blood pressure cuff or the like. The infusion device 13 typically delivers a steady infusion rate of metoprolol to the patient, with the rate selected to match the prior dosage of metoprolol (or other beta-blocker) that the patient was taking orally prior to hospitalization. While the actual patient dosing schedule would be determined by the patient's physician and would be based on clinical data, one such exemplary schedule is provided below for illustrative purposes as Table 1:

| | | | | | Loading | Continous |
|---|---|---|---|---|---|---|
| | | | | | Dose | Infusion |
| | | | | | IV | IV |
| Oral Medications | | | | | Metoprolol | Metoprolol |
| Atenolol | Carvedilol | Coreg CR | Metoprolol | Toprol XL | Bolus | Infusion |
| 25 mg PO | 3.125/6.25 | 10/20 mg | 25 mg PO | 50 mg PO daily | 5 mg | 2 mg/hr |
| daily | PO bid | PO daily | bid | | | |
| 50 mg PO | 12.5 mg PO | 40 mg PO | 50 mg PO | 100 mg PO daily | 10 mg | 4 mg/hr |
| daily | bid | daily | bid | | | |
| 100 mg | 25 mg PO | 80 mg PO | 100 mg PO | 200 mg PO daily | 15 mg | 6 mg/hr |
| PO daily | bid | daily | bid | | | |

Alternatively, the infusion device 13 may be set to deliver an empirically or clinically determined rate of infusion if the patient's prior dose of beta blocker is unknown or if the patient has not been on a beta blocker agent previously.

A nurse or technician records and reviews the results of the heart rate and blood pressure values, such as in a paper chart, electronic medical record, or the like. Subsequently, through an order set, written algorithm, computer program, calculator or the like, the nurse makes adjustments to the infusion device 13 to correct the dose of metoprolol infusion based on patient's hemodynamic and clinical status.

In a second embodiment, the system 10 is similar to the one discussed above, but with heart rate and blood pressure sensors 26, 28 connected, either directly or wirelessly, to a telemetry or hemodynamic monitoring system 18. The nurse records and reviews the results of the heart rate and blood pressure values, either in a paper chart, electronic medical record or the like. Subsequently, typically through an order set, written algorithm, computer program, calculator or like, the nurse makes adjustments to the infusion device 13 to correct the dose of metoprolol infusion based on patient's hemodynamic and clinical status.

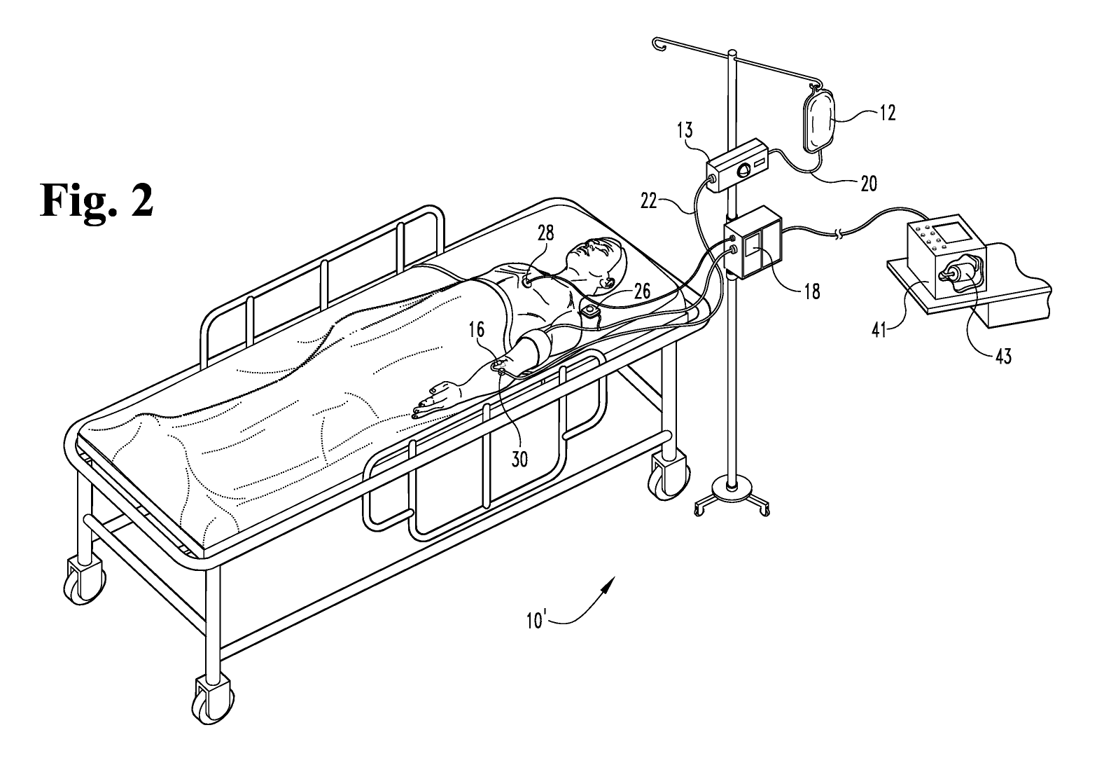

In a third embodiment system 10′, as shown in FIG. 2, the system 10′ is similar to the first and second embodiments discussed above, but with the heart rate and blood pressure sensors 26, 28 connected either directly or wirelessly to a telemetry or hemodynamic monitoring system 18, which is connected to send the heart rate and blood pressure values to the patient's electronic medical record 41. A nurse or technician reviews the results in the electronic medical record and subsequently, such as through an order set, written algorithm, computer program, calculator or like, makes adjustments to the infusion device 13 to correct the dose of metoprolol infusion based on patient's hemodynamic and clinical status.

In a fourth embodiment system 10′, the system 10′ is similar to those discussed above but with the heart rate and blood pressure sensors 26, 28 connected, either directly or wirelessly, to a telemetry or hemodynamic monitoring system 18 which is connected to send the heart rate and blood pressure values directly into the patient's electronic medical record 41. The electronic medical record 41 is operationally connected to a microprocessor 43 which applies the hemodynamic data to an internal computer program, which calculates and provides directions to the nurse on making adjustments to the dose of metoprolol infusion, based on the patient's hemodynamic and clinical status.

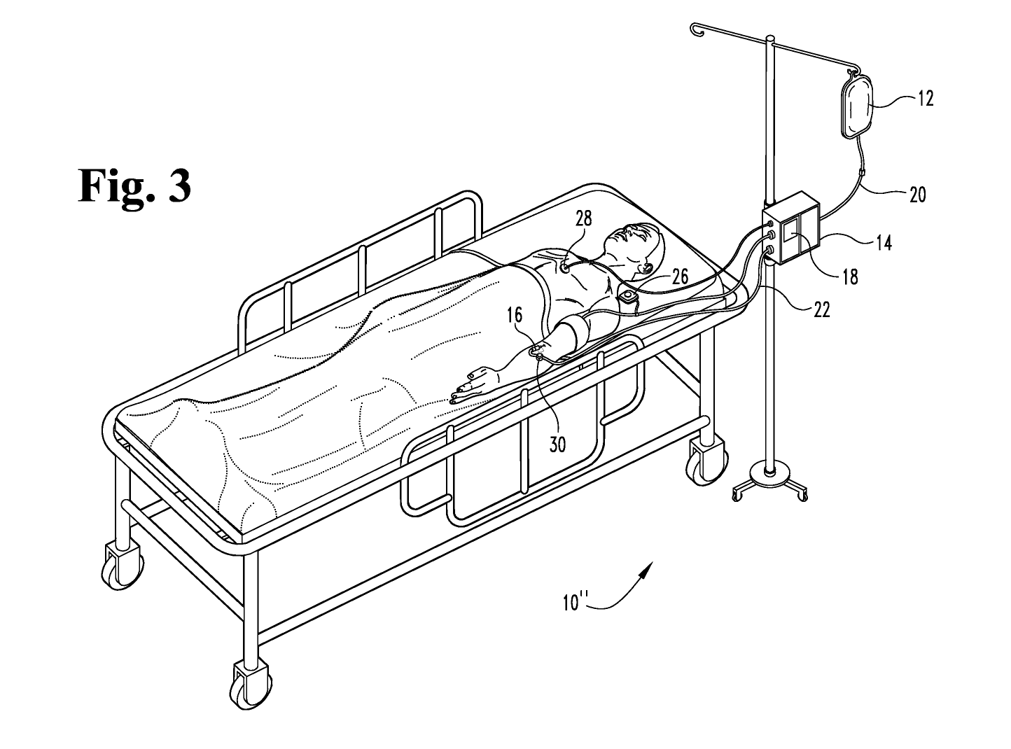

A fifth embodiment system 10″, as shown in FIG. 3, includes a metoprolol supply 12 in liquid communication with an infusion pump 14. The pump 14 is connected in liquid communication with catheter 16 or like device for insertion into a patient's circulatory system. The pump 14 is operationally connected to an electronic controller 18 for governing the flow rate of the metoprolol through the catheter 16.

The metoprolol supply 12 is typically connected to the pump 14 via first tubing 20, and the pump 14 is typically connected to the catheter 16 via second tubing 22. Typically, a first sensor 26 is operationally connected to the electronic controller 18. The first sensor 26 may be a blood pressure sensor, for example, and is operationally connectable to the patient for providing substantially real time blood pressure data to the electronic controller 18.

More typically, a second sensor 28, such as for measuring a patient's heart rate, is operationally connected to the electronic controller 18 and is likewise operationally connectable to the patient for providing substantially real-time heart rate data to the electronic controller 18. Still more typically, a third sensor 30, such as for directly measuring the concentration of metoprolol in the patient's blood, is operationally connected to the electronic controller 18 and is likewise operationally connectable to the patient for providing substantially real-time metoprolol concentration data to the electronic controller 18.

Metoprolol source 12 is typically a 50 or 100 mg bag, although bags or containers of other sizes may be selected as desired. Pump 14 is typically a standard intravenous pump as is known in the art. Catheter 16 may be of any convenient vascular access device design for insertion into a central or peripheral vein.

The metoprolol source 12 may contain a standard concentration of metoprolol, akin to that found in 5 mg single-dosage vials (typically 1 mg/ml), or, alternately, may contain a metoprolol solution having a lower (or greater) metoprolol concentration. In one embodiment, the electronic controller 18 may be configured to control the pump 14 to provide a predetermined, steady infusion rate of metoprolol to the patient, with the rate selected to match the prior dosage of metoprolol (or other beta-blocker) that the patient was taking orally prior to hospitalization. While the actual patient dosing schedule would be determined by the patient's physician and would be based on clinical data, one such exemplary schedule is provided below for illustrative purposes as Table 1:

| | | | | | Loading | Continous |
|---|---|---|---|---|---|---|
| | | | | | Dose | Infusion |
| | | | | | IV | IV |
| Oral Medications | | | | | Metoprolol | Metoprolol |
| Atenolol | Carvedilol | Coreg CR | Metoprolol | Toprol XL | Bolus | Infusion |
| 25 mg PO | 3.125/6.25 | 10/20 mg | 25 mg PO | 50 mg PO daily | 5 mg | 2 mg/hr |
| daily | PO bid | PO daily | bid | | | |
| 50 mg PO | 12.5 mg PO | 40 mg PO | 50 mg PO | 100 mg PO daily | 10 mg | 4 mg/hr |
| daily | bid | daily | bid | | | |
| 100 mg | 25 mg PO | 80 mg PO | 100 mg PO | 200 mg PO daily | 15 mg | 6 mg/hr |
| PO daily | bid | daily | bid | | | |

Alternatively, the electronic controller 18 may be configured to control the pump 14 and deliver an empirically or clinically determined rate of infusion if the patient's prior dose of beta blocker is unknown or if the patient has not been on a beta blocker agent previously.

In a sixth embodiment system 10″, the electronic controller 18 is configured to control the pump 14 to deliver metoprolol at a default flow rate similar to that described above, and is also configured to vary the flow rate based on data provided by one or more of the sensors 26, 28, 30. The electronic controller 18 is programmed to decrease the flow of metoprolol if the data received from the sensors 26, 28, 30 indicates that the patient has received an excess dosage, and to increase the flow of metoprolol if the data indicates that the patient has not yet received a sufficient dosage. In other words, the electronic controller 18 is programmed to compare the data received from the sensors 26, 28, 30 with predetermined parameters, such as tabulated data, mathematical relationships, or the like, and vary the flow of metoprolol according to the results. For example, if the patient's blood pressure and/or heart rate are too high, the flow of metoprolol may be incrementally increased until either a maximum cap flow rate is achieved or the measured blood pressure and/or heart rate decreases below a predetermined level.

In a seventh embodiment system 10″, the electronic controller 18 is configured to control the pump 14 to deliver metoprolol at a default flow rate similar to that described above, and is also configured to compare the data received from the sensors 26, 28, 30 with predetermined parameters, such as tabulated data, mathematical relationships, or the like, to calculate one or more recommended courses of action based on the same, and to display the calculated course(s) of action to the appropriate medical personnel. For example, if the patient's blood pressure and/or heart rate are too high, the electronic controller 18 may display a recommendation that the flow of metoprolol be increased by a predetermined amount so that the measured blood pressure and/or heart rate decreases below a predetermined level. The electronic controller 18 may also recommend other medications, procedures, physician attendance to the patient, or the like.

In an eighth embodiment system 10″, the electronic controller 18 may be configured to generate an alert signal if a patient's heart rate and/or blood pressure and/or other like physical conditions, as measured by sensors 26, 28, 30 fall outside of predetermined parameters. Such a signal would alert hospital personnel to manually change the flow rate of the metoprolol and/or take whatever other steps necessary to bring the patient's physical metrics (i.e., heart rate and/or blood pressure and the like) back into the predetermined parameters.

In a ninth embodiment system 10″, the electronic controller 18 is configured to both vary the flow rate of metoprolol to keep the patient's blood pressure and/or heart rate and the like, as measured by sensors 26, 28, 30 within a predetermined set of parameters and to also generate an alert signal when the patient's blood pressure and/or heart rate or the like falls outside the scope of those measured parameters.

It should be recognized that the above embodiments are not mutually exclusive.

While the invention has been illustrated and described in detail in the drawings and foregoing description, the same is to be considered as illustrative and not restrictive in character. It is understood that the embodiments have been shown and described in the foregoing specification in satisfaction of the best mode and enablement requirements. It is understood that one of ordinary skill in the art could readily make a nigh-infinite number of insubstantial changes and modifications to the above-described embodiments and that it would be impractical to attempt to describe all such embodiment variations in the present specification. Accordingly, it is understood that all changes and modifications that come within the spirit of the invention are desired to be protected.

What is claimed is:

1. A system for automatically providing a continuous flow of metoprolol intravenously to a patient, comprising:
a supply of metoprolol solution;
a pump connected in fluidic communication with the supply of metoprolol solution;
an electronic controller operationally connected to the pump;
a vascular access device connected in liquid communication to the pump; and
at least one sensor operationally connected to the pump;
wherein the pump provides a continuous flow of metoprolol through the vascular access device;
wherein the at least one sensor provides data regarding a patient's physical condition to the electronic controller;
wherein the electronic controller may control the pump to vary the flow rate of metoprolol in response to data from the at least one sensor; and
wherein the electronic controller may generate an alarm signal in response to data from the sensor falling outside of predetermined parameters.

2. The system of claim 1 wherein the at least one sensor is a blood pressure sensor.

3. The system of claim 1 wherein the at least one sensor is a heart rate sensor.

4. The system of claim 1 wherein the electronic controller incrementally varies the flow of metoprolol through the vascular access device until the data from the at least one sensor is within a set of predetermined parameters.

5. The system of claim 1 wherein the electronic controller displays a recommended course of action when the data from the at least one sensor is outside a set of predetermined parameters.

6. The system of claim 1 wherein the vascular access device is a catheter.

7. A method for automatically administering metoprolol to a patient to control heart rate and/or blood pressure, comprising:
a) supplying a quantity of liquid metoprolol solution to an infusion device;
b) connecting the infusion device in fluidic communication with a patient's circulatory system;
c) measuring a physical characteristic of the patient selected from the group including blood pressure, heart rate, blood metoprolol concentration, and combinations thereof;
d) flowing metoprolol into the patient's circulatory system at a predetermined rate;
e) comparing the measured physical characteristics of the patient to predetermined parameters; and
f) varying the rate of metoprolol flow into the patient's circulatory system if the measured physical characteristics are outside of the predetermined parameters.

8. The method of claim 7 wherein the flow of metoprolol is automatically decreased if the patient's heart rate is too low.

9. The method of claim 7 wherein the flow of metoprolol is automatically decreased if the patient's blood pressure is too low.

10. The method of claim 7 wherein the flow of metoprolol is automatically increased if the patient's heart rate is too high.

11. The method of claim 7 wherein the flow of metoprolol is automatically increased if the patient's blood pressure is too high.

12. The method of claim 7 and further comprising:
g) generating an alarm signal if the measured physical characteristics are outside of the predetermined parameters.

13. The method of claim 7 and further comprising:
h) automatically recommending a course of action if the measured physical characteristics are outside of the predetermined parameters.

14. The method of claim 7 further comprising i) connecting a microprocessor to the infusion device and j) operationally connecting blood pressure and heart rate sensors to the patient and to the microprocessor; wherein the infusion device is a pump; and wherein steps c), d), e) and f) are controlled by the microprocessor.

15. A system for governing the continuous flow of metoprolol intravenously to a patient, comprising:
a supply of standardized metoprolol solution;
an infusion device connected in fluidic communication with the supply of metoprolol solution;
a vascular access device connected in liquid communication to the infusion device; and
at least one sensor operationally connected to the patient;
wherein the infusion device regulates a continuous flow of metoprolol through the vascular access device;
wherein the at least one sensor provides data regarding a patient's physical condition to at least one observer; and
wherein the at least one observer may control the infusion device to vary the flow rate of metoprolol in response to data from the at least one sensor.

16. The system of claim 15 wherein the at least one observer is a microprocessor operationally connected to the infusion device and to the at least one sensor.

17. The system of claim 16 wherein the infusion device is a pump.

18. The system of claim 16 wherein the at least one observer includes member of the set containing the patient's electronic medical records, a nurse, a microprocessor, and combinations thereof.
